(19) United States
(12) Patent Application Publication
(10) Pub. No.: US 2023/0285711 A1
(43) Pub. Date: Sep. 14, 2023

(54) Assessing User Engagement to Optimize the Efficacy of a Digital Mental Health Intervention

(71) Applicant: Koa Health B.V., Barcelona (ES)

(72) Inventors: Albert Garcia i Tormo, Barcelona (ES); Nicola Hemmings, Bristol (GB); Claire Vowell, Shaw Hill (GB); Teodora Sandra Buda, Barcelona (ES); Remko Vermeulen, Barcelona (ES)

(21) Appl. No.: 17/835,581

(22) Filed: Jun. 8, 2022

(30) Foreign Application Priority Data
Mar. 14, 2022 (EP) 22161968.7

(51) Int. Cl. A61M 21/00; G16H 20/70; G16H 40/63

(52) U.S. Cl. CPC A61M 21/00; G16H 20/70; G16H 40/63; A61M 2021/0027; A61M 2021/005

(57) ABSTRACT

A method determines the most effective motivator at inducing a user to engage in a digital mental health intervention. The user is exposed to a first motivator that prompts the user to perform the intervention. The motivator can be a video, audio tape, textual explanation or quiz-like game. Intervention and motivator parameters are monitored to assess user engagement both with the first motivator and in performing the intervention. An intervention delivery model is personalized to the user based on both parameters. The intervention delivery model is used to determine the efficacy of the first motivator at motivating the user to perform the intervention. The intervention and motivator parameters are compared to an intervention engagement threshold and a motivator engagement threshold. If either or both parameters are below the corresponding threshold, the intervention delivery model is used to select a second motivator. The user is then exposed to the second motivator.

### CROSS REFERENCE TO RELATED APPLICATION

This application is based on and hereby claims the benefit under 35 U.S.C. § 119 from European Patent Application No. EP 22161968.7, filed on Mar. 14, 2022, in the European Patent Office. This application is a continuation-in-part of European Patent Application No. EP 22161968.7, the contents of which are incorporated herein by reference.

### TECHNICAL FIELD

The present invention relates to a customizable therapy delivery system that optimizes a user's engagement in interventions that are part of digital mental health therapies.

### BACKGROUND

Due to the increasing digitalization and automatization in our modern world, ensuring an efficient interaction between human users and a variety of devices, such as factory machines and electronic consumer devices, becomes increasingly important. In this context, ensuring user engagement with the device has emerged as one of the main challenges in ensuring an efficient and stable machine-user interaction. A similar consideration is ensuring that a user of an electronic device that delivers a cognitive behavioral therapy is engaged with the device and the therapy. In practice, often users do not feel engaged and feel reluctant to interact with the electronic device or machine and may even eventually stop interacting with the device altogether.

Previous attempts to improve user engagement have generally focused on designing user friendly application interfaces or on adjusting the content delivered via the device. For example, if the user repeatedly fails to perform maintenance work on a machine or device after the machine has issued a corresponding alert, the machine may issue an instruction to contact a maintenance worker based on the assumption that the user might at least be capable of performing this simpler task.

However, previous solutions have focused mainly on adjusting the content (the “what”) delivered by the device in order to maximize user engagement, thereby delivering the same content in the same way to all users, regardless of the characteristics of the individual user. Individual users, however, may differ significantly from each other and may respond to different modes of interaction differently.

In an analogy to sports, the activity for which user engagement is to be improved is mastering a punching technique in martial arts. Some students may learn well by carefully observing the instructor (focusing on the position of the legs and movement of the arms, shoulders and hips) and practicing on their own. Other students may need to practice with a punching bag to feel the resistance when properly coordinating the movement. Yet other students may need the instructor or a colleague to help them position themselves in the correct intermediate positions.

In all cases, the “what” is the same: learning the punching technique. But the “how”, the manner in which the user learns and engages in the martial arts activity, differs. All students could probably learn the punching technique to some extent by using any of the teaching/learning methods. But for each individual there is one method that allows the user to learn faster or to achieve a better mastery of the technique. Moreover, depending on the moment, the learning method might need to be changed in order to continue improving. The challenge is, for each student and each moment in time, to identify the most effective learning method in order to improve the punching technique.

Customizing the manner in which content is delivered (the “how”), however, typically remains unexplored in the context of human-machine interaction. Differing methods for delivering content allow user engagement to be enhanced in different ways.

Thus, there exists a need for an improved method by which an individual user is induced to interact in a particular way with a device or to engage in a desired manner in an activity or mental health intervention that optimizes the user's engagement with the device or activity. In addition, there exists a need for a machine capable of interacting with a user in a manner that induces the user to successfully complete the required maintenance work and not simply to compete the simpler task of calling a maintenance worker.

### SUMMARY

The present invention relates to a device or machine that uses a software model to automatically tailor the communication mode between the machine and the user to the individual characteristics of the user without the user having to provide active input regarding the user's preferences. Instead, the model selects the most suitable motivator to induce the user to engage in performing an instructed activity. For example, the activity is a step of controlling the machine in a complex manufacturing process. Thus, conscious biases of the user, such as the user stating a preference for written text but in fact being more responsive to videos, do not hamper the interaction of the user with the machine, and the efficacy of the machine-user interaction is improved.

In one embodiment, a method for assessing how engaged a user is in an activity determines the efficacy of a motivator at inducing the user to perform the activity. The user is instructed to perform the activity. An activity parameter is monitored to assess the engagement of the user in performing the activity. The user is exposed to a first motivator that prompts the user to perform the activity. A motivator parameter is monitored to assess the engagement of the user with the first motivator. The efficacy of the first motivator at motivating the user to perform the activity is determined using a model customized to the user based on the activity parameter and the motivator parameter. The activity parameter is compared to an activity engagement threshold. The motivator parameter is compared to a motivator engagement threshold. If either or both the activity parameter is below the activity engagement threshold or the motivator parameter is below the motivator engagement threshold, the model is used to select a second motivator. The user is then exposed to the second motivator.

An attribute of the user is identified using the model that indicates a preference of the user for the first motivator. Based on the attribute, a support source is selected that the model predicts will likely support the user in performing the activity. The support source can be a physician, a health professional, a chatbot, or an avatar.

In another embodiment, a system for assessing how engaged a user is in an activity includes a monitoring unit that assesses the engagement of the user with a motivator. An output unit instructs the user to perform the activity. The monitoring unit assesses the engagement of the user in performing the activity by monitoring an activity parameter. The output unit exposes the user to a first motivator that prompts the user to perform the activity. The monitoring unit assesses the engagement of the user with the first motivator by monitoring a motivator parameter. An evaluation unit determines the efficacy of the first motivator at motivating the user to perform the activity by using a model customized to the user based on the activity parameter and the motivator parameter. A comparison unit compares the activity parameter to an activity engagement threshold and compares the motivator parameter to a motivator engagement threshold. The evaluation unit uses the model to select a second motivator if either or both the activity parameter is below the activity engagement threshold or the motivator parameter is below the motivator engagement threshold. The output unit exposes then the user to the second motivator.

In yet another embodiment, a method for assessing how engaged a patient is in a digital mental health intervention determines the most effective motivator to induce the patient to engage in the intervention. The patient is exposed to a first motivator that prompts the patient to perform the intervention. The first motivator can be watching a motivational video, listening to a motivational audio tape, engaging in a quiz-like game, or reading an explanation of how the intervention will benefit the patient. An intervention parameter is monitored to assess the engagement of the patient in performing the intervention. A motivator parameter is monitored to assess the engagement of the patient with the first motivator. An intervention delivery model is personalized to the patient based on the intervention parameter and the motivator parameter. The efficacy of the first motivator at motivating the patient to perform the intervention is determined using the intervention delivery model. The intervention parameter is compared to an intervention engagement threshold, and the motivator parameter is compared to a motivator engagement threshold. If either or both the intervention parameter is below the intervention engagement threshold or the motivator parameter is below the motivator engagement threshold, the intervention delivery model is used to select a second motivator. The patient is then exposed to the second motivator.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 shows a preferred embodiment of the invention focusing in particular on an embodiment of a model used by the present invention.

FIG. 2 shows another embodiment that uses motivators to induce a patient to attempt to complete a therapy or intervention.

FIG. 3 shows an example of engagement evaluation based on an exponential model.

FIG. 4 is a table showing an example of evaluating current user engagement based on an exponential model.

FIG. 5 shows an example of a breathing signal measured with a camera (upper plot) and the inhalations and exhalations identified from the derivative signal (lower plot) by comparing to a threshold.

FIG. 6 is a table showing exemplary scenarios relating to user engagement in an activity induced by a motivator and actions recommended by the model.

### DETAILED DESCRIPTION

Reference will now be made in detail to some embodiments of the invention, examples of which are illustrated in the accompanying drawings.

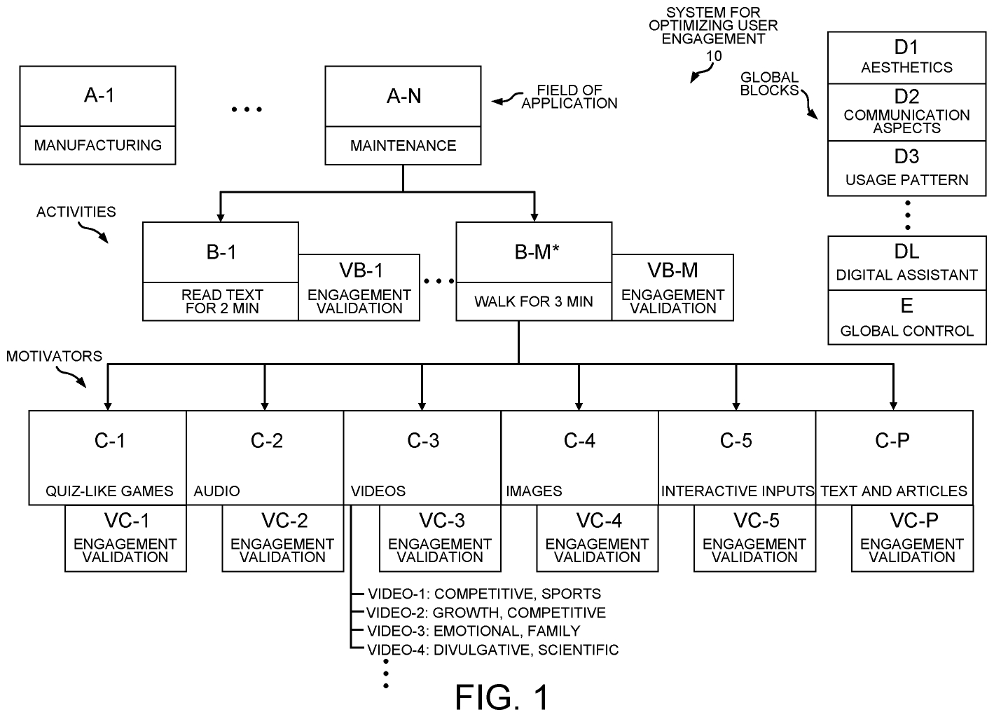

FIG. 1 shows a system 10 for enhancing user engagement with a machine or device. The system 10 implements a method for assessing and/or optimizing the engagement of the user in at least one activity and is configured to:

instruct the user to perform at least one activity by means of an output unit,

monitor at least one first parameter by means of a monitoring unit to assess the engagement of the user in performing the at least one activity or attempting to perform the at least one activity,

expose the user to at least one first motivator by means of the output unit, wherein the motivator is chosen to prompt or motivate the user to perform the at least one activity or to attempt to perform the at least one activity,

monitor at least one second parameter by means of the monitoring unit to assess the engagement of the user in the at least one motivator,

enter the first and second parameters into a model of the user that indicates the efficacy of at least one motivator for motivating the user to perform or to attempt to perform at least one activity, wherein the model is preferably customized to the individual user,

compare by means of a comparison unit the at least one first parameter or an activity engagement score based on the at least one first parameter to a threshold value, and/or

compare by means of the comparison unit the at least one second parameter or an motivator engagement score based on the at least one second parameter to a threshold value,

if the comparison indicates that the at least one first parameter or the activity engagement score and/or the at least one second parameter or the motivator engagement score is below the threshold value,

select by means of the model at least one second motivator from a list of motivators accessible by the model, and

by means of a control unit control the device to expose the user to the least one second motivator by means of the output unit.

In one embodiment, the device is a machine used in manufacturing, and the user is required to interact with the machine with a high degree of engagement to control the machine during a complex manufacturing process. Initially, the machine may issue an instruction to the user via an output unit to instruct the user to walk to a storage room and fetch a required component.

For example, by means of a wearable device that captures accelerometer data, it can be assessed how much time the user spends walking. This gives an indication of the time the user spends performing the instructed activity or at least in attempting to do so.

To motivate the user to successfully perform the instructed activity, the machine exposes the user to a motivator, for example, a text appearing on a screen of the machine explaining why the component to be fetched is important for the subsequent manufacturing process.

The motivator may be issued simultaneously with the instruction to perform the activity or with a delay. The engagement of the user with the motivator is assessed, e.g., by measuring the time the user spends reading the text.

The data indicating the engagement of the user in the activity, in this example the time the user spent walking, and the data indicating the engagement of the user with the motivator, in this example the time the user spent reading the text, are then input into a model of the user that indicates that this particular user is reasonably responsive to motivators in the form of text but is much more responsive to motivators in the form of videos.

It is then assessed whether the engagement of the user in the activity is satisfactory. For example, the time the user spent walking (e.g., 2 min) is compared to the time it should take the user to fetch the component from the storage room (e.g., 5 min). The measured walking time may be compared directly to a threshold value, for example, the measured 2 minutes of walking may be compared to 4 minutes corresponding to the time even a very fast walker would require to fetch the component.

Alternatively, a quotient may be calculated, for example 2 minutes measured activity out of 5 minutes required activity, corresponding to a quotient of 2/5=0.4. This quotient 0.4 may then be compared to a threshold value of 2/4=0.5 indicating a very fast walker successfully completing the activity.

The time the user spent engaged with the motivator, i.e., the time the user spent reading the text may alternatively or additionally be compared directly to a threshold value or a score, such as a quotient may be calculated that is then compared to a threshold value.

If the comparison with the threshold value indicates that the user did not perform the activity satisfactorily, e.g., the user did not walk far enough, and/or the user did not engage satisfactorily with the motivator, e.g., the user read the text for only 30 seconds even through one minute would be required for attentive reading, the model is used to select at least one different motivator, such as a video illustrating the instructed activity or a video explaining why the required component is important. For example, in the past, the user has shown to be responsive to videos.

In this case, the model selects a video as an alternative or additional motivator. The control unit of the machine then controls the machine to display to the user the video selected by the model.

Thus, a device according to the present invention tailors the communication mode with a user to the individual characteristics of the user without the user having to provide active input regarding the user's preferences. Instead, the model selects the most suitable motivator for the user. Thus, conscious biases of the user, such as the user stating that he prefers written text but in fact being more responsive to videos, do not hamper the interaction of the user with the device. In other words, a system according to the invention preferably phrases instructions to the user (e.g., by choosing suitable motivators) in a way that causes the user to be optimally responsive.

The application described above is only one example, and the present invention may be also used for other exemplary applications, such as to encourage an employee to follow an online course on cyber security or another topic or to attentively follow a manual or video detailing how to operate a given machine. Also, in order to ensure safety of the user, the user may be encouraged to take, e.g., three breaks from work each day and spend these breaks walking to increase productivity and concentration of the user, e.g., if the user has to guide a machine through a complex process over several hours.

Generally, the tasks or instructed activities may be set by the users themselves (the users thus perform the instructed tasks voluntarily) and/or by other persons and/or devices.

In one embodiment, the device is configured to continue selecting other motivators until the comparison indicates that at least one first parameter or the activity engagement score and/or at least one second parameter or the motivator engagement score meets or surpasses the threshold value. In other words, the device may be configured to prompt the user with different motivators until a desired level of engagement of the user in an instructed activity and/or with a motivator has been achieved.

If the device detects that the first parameter, the activity engagement score, the second parameter, and/or the motivator engagement score fails to meet or surpass the threshold value for a defined period of time, the device may be configured to notify an external source, e.g., an external device, accordingly. The device may issue a notification to another device that may be of a higher instance or person, for example a device or person that set the instructed activity.

In this way, in cases that the user is highly engaged but fails to complete the task in the defined period of time or user engagement with the device cannot be achieved to a satisfactory level in the defined period of time, a notification or report is issued to a higher level.

The defined period of time may be set by default, by the person or device setting the instructed activity or may be automatically set by the device, preferably in an individualized way for each user instructed to perform a task.

In one embodiment, the threshold is predefined, is a default value or is determined for each individual user.

The device may be configured to instruct multiple activities and to induce these activities each with a different motivator that is particularly suitable for increasing user engagement with that activity.

In one embodiment, the model implemented by system 10 includes a plurality of task elements for each field of application. In FIG. 1, the task elements are illustrated as elements A. Each task element is associated with a plurality of activity elements, which are illustrated in FIG. 1 as elements B and which correspond to different activities that the user may be instructed to perform in the course of completing a task.

In one embodiment, each of the activity elements B includes a designated validation element, which are denoted in FIG. 1 as elements VB. The validation elements of the model shown in FIG. 1 are configured to assess the engagement of the user in the activity corresponding to the activity element.

The model implemented by system 10 also includes, for each activity element, a plurality of motivation elements, which are denoted in FIG. 1 as elements C. Each motivation element includes a designated validation element, which are denoted in FIG. 1 as elements VC. The validation elements are configured to assess the engagement of the user in the motivation element corresponding to the activity element. A motivation element is also called a motivator.

The model includes, for each motivation element, a plurality of motivation specimens, wherein the motivation element defines a group into which all the motivation specimens associated with this motivation element fall. For example, in the model shown in FIG. 1, videos video-1 through video-4 are motivation specimens associated with the motivation element C-3 that defines the group “videos”.

In one embodiment, the model implemented by system 10 is continuously updated based on parameters monitored by the monitoring unit, such as the first and second parameters entered into the model, and/or calculated values derived from these parameters. This has the benefit that the model constantly learns about changing characteristics and preferences of the user and thereby generates a constantly accurate and up-to-date selection of the most suitable motivator(s).

The model is automatically updated without any specific input to that effect from the user. A device on which the novel method is implemented analyzes the interaction of the user with the device and/or the behavior of the user and/or passively acquires data relating to the user and thus is capable of optimizing the communication of the device with the user without the user having to actively provide input to that effect. The device is configured to by means of an evaluation unit to evaluate for a past time interval a change in the first parameter, the activity engagement score, the second parameter, and/or the motivator engagement score.

The past time interval may, for example, be the last hour, the last day, the last week, the last year or any other past time interval. The time interval may also span the time between two attempts of the user at completing the instructed activity and/or between two instances of interaction of the user with the device. For example, the novel method evaluates how the engagement of the user in the instructed activity and/or the engagement of the user with the motivator may change with time.

According to an embodiment, a device is configured based on parameters monitored by the monitoring unit, such as the first and second parameters entered into the model, and/or calculated values derived from these parameters. The model is then used to identify an attribute of the user that characterizes the user and in particular indicates a preference of the user for at least one motivator.

For example, the model might determine that the user often clicks on a “learn-more” button present on a graphical user interface of the device in order to learn more about an instructed activity. From this, the model can derive that the user is relatively curious and/or that the user performs activities more reliably if the user understands the reason for the activity. Thus, the model may select a motivator that exposes the user to interesting background information about an instructed activity to induce the user to more reliably perform the instructed activity.

According to another embodiment, the device is configured by means of the model to select from a stored list of support sources, based on an attribute of the user, a support source expected to optimally support the user in performing the activity or attempting to perform the activity, wherein the support source preferably is a person or a virtual support system, such as a chat bot or an avatar. For example, if the model has determined that the user is relatively sociable, the model selects a friendly avatar to be displayed by the device on a graphical user interface to the user to guide the user through the steps of, e.g., a complex manufacturing process to be performed by the device. If the model, however, determines that based on the user's age and behavior, the user is likely to be more comfortable with a book than with a graphical user interface, the device may draw the user's attention to a physical manual. When selecting a support source the device may draw on a data base of possible support sources.

In case of virtual support systems, such as chat bots or avatars, the device may retrieve a suitable chat bot or avatar from a database of chat bots and avatars.

In order to further improve user engagement, the device can be configured to generate based on an attribute of the user a virtual support system, e.g., based on the model, that is expected to optimally support the user in performing the activity or attempting to perform the activity, wherein the virtual support system is a chat bot or an avatar. In other words, if no optimally suitable chat bot or avatar for a particular user can be retrieved from the database, the device can generate a customized virtual support system, such as a chat bot or an avatar, for the user. For this effect, an existing chat bot or avatar may be modified or a new chat bot or avatar can be generated from scratch.

In one embodiment, the device is configured to instruct the user to perform a plurality of activities by means of the output unit, and for each activity of the plurality of activities expose the user to at least one first motivator, wherein the motivators may differ from each other. For example, the user may be instructed to walk to the storage room to fetch a component and to keep an eye on a temperature sensor. For the first activity, the user may be most responsive to a text providing background information on the importance of the component, and for the second activity the user may be most responsive when exposed to a video illustrating the effects of over-heating.

According to an embodiment, different motivators are configured to convey the same content to the user in different formats and/or modes of delivery. For example, one motivator may be a text providing background information on why a component is required, and a second motivator may be a video providing background information on why the component is required.

Another aspect of the invention relates to a method for assessing and/or optimizing the engagement of a user in an activity, the method preferably being performed by a device or machine according to the invention. The method involves:

instructing the user to perform an activity, preferably by using an output unit,

monitoring a first parameter using a monitoring unit to assess the engagement of the user in performing the activity or attempting to perform the activity,

exposing the user to a first motivator, preferably using the output unit, wherein the motivator is chosen to prompt or motivate the user to perform the activity or to attempt to perform the activity,

monitoring a second parameter, preferably using the monitoring unit, to assess the engagement of the user with the first motivator,

entering the first and second parameters into a model of the user that indicates the efficacy of the first motivator at motivating the user to perform or to attempt to perform the activity, wherein the model is preferably customized to the individual user,

comparing, preferably using a comparison unit, the first parameter or an activity engagement score based on the first parameter to a threshold value, and/or

comparing, preferably using the comparison unit, the second parameter or a motivator engagement score based on the second parameter to a threshold value,

if the comparison indicates that the first parameter or the activity engagement score and/or the second parameter or the motivator engagement score is below the threshold value, selecting by means of the model a second motivator from a list of motivators accessible by the model, and

exposing the user, preferably using a control unit controlling the device, to the second motivator, preferably using the output unit.

The model is continuously updated based on parameters monitored by the monitoring unit, such as the first and second parameters entered into the model, and/or calculated values derived from these parameters.

The method also involves the step of evaluating for a past time interval, preferably using an evaluation unit, a change in the first parameter or the activity engagement score and/or the second parameter or the motivator engagement score.

According to an embodiment, the method also includes the step: based on parameters monitored preferably by the monitoring unit, such as the first and second parameters entered into the model, and/or calculated values derived from these parameters, identifying by means of the model at least one attribute of the user that characterizes the user and in particular indicates a preference of the user for at least one motivator.

The method is performed automatically, preferably without the user being required to provide specific input beyond the interaction of the user with the device.

According to another embodiment, the method also involves, by means of the model, selecting from a stored list of support sources based on the attribute of the user a support source expected to optimally support the user in performing the activity or attempting to perform the activity, wherein the support source preferably is a person or a virtual support system, such as a chat bot or an avatar.

The model used in the novel method includes the same features as a model described in the context of a device or machine according to the invention.

The method involves generating, based on the attribute of the user, a virtual support system expected to optimally support the user in performing the activity or attempting to perform the activity, wherein the virtual support system is a chat bot or an avatar.

According to another embodiment, the method also involves instructing the user to perform a plurality of activities using the output unit, and for each activity of the plurality of activities exposing the user to one or more first motivators, wherein each of the first motivators may differ from the others. Preferably, different motivators are configured to convey the same content to the user in different formats and/or modes of delivery. When a user interacts with a device, it often is necessary to induce the user to perform a specific action or activity. Thus, the device is preferably controlled to induce the user to perform a defined action or activity and to ensure satisfactory user engagement, e.g., for the user to follow through with completing the instructed activity. In other words, something is defined that the user must do (i.e., the what). However, the way of reaching out to the user, that is, how to make sure that the user understands what he or she needs to do and to make the user do it and engage in the instructed, is something that can be done in different ways.

For example, some users may engage in a quiz-like game, as a challenging or competitive activity. Other users may dislike the competitive aspect and may prefer to watch a video. Despite using different ways of reaching, motivating and involving the user, in both cases, quiz and video, the goal is the same: to induce the user to perform a specific activity or action. In other words, preferably the same instruction (i.e., same what) is delivered in different ways (different how) tailored to individual users.

An action or activity can be motivated and/or delivered in multiple ways, including quiz-like games, images, audio content, video content, interactive inputs and texts and articles. Each of the instructions can hence be delivered in any of these ways, which unfolds a wide range of possibilities of reaching users and keeping them engaged.

The novel method accomplishes the objective of improving user engagement in the interaction of the user with a device or machine by optimizing the way in which an instruction is delivered in order to maximize its effectiveness. The most effective way of delivering an instruction is not necessarily the one that the user would consciously choose or claim to feel most comfortable with. The objective of the novel method is to find the most effective way to deliver an instruction to a user (in other words, to reach the user) and to ensure that the user is motivated to perform the instructed action.

The novel method automatically adjusts the how, the manner in which the instruction is delivered to the user, in order to maximize user engagement. Maximizing user engagement requires distinguishing that which is preferred by the user from that which is effective for the user. Adjusting the how (or personalizing the delivery of the instruction) involves not only adapting a variety of aspects such as the aesthetics of a graphical user interface presented to the user, the mode or way of interacting with the device (e.g., voice or text, for both input and output) or the amount of contextual information provided by default, but also adapting the format in which the instruction is delivered (e.g., in the form of quiz-like games, via audio content, video content, images, interactive inputs or texts).

Likewise, personalizing the delivery preferably requires monitoring several aspects of the user, including both behavior-based aspects and physiological-based aspects. These aspects are monitored by the monitoring unit using sensors. The measurements preferably are based on real-time resolution data (inputs that are gathered frequently, from daily activities, sometimes even in the moment), in order to distinguish that which is effective for the user from that which is preferred by the user.

Achieving such personalization in a non-automatic manner has disadvantages because gathering daily inputs from the user requires the continuous supervision of the user to assess the user's behavior. Even if continuous supervision of the user were feasible, the observed user's behavior would be conditioned by the fact that another person would be supervising the user, resulting in a biased measurement.

Thus, parameters of the user are preferably automatically monitored by a monitoring unit without the user noticing and/or without the user having to actively provide input. The monitoring unit may include several sensors, such as proximity sensors, GPS and location sensors, gyrometers, accelerometers, a camera, a microphone, etc. The monitoring unit may also include portable devices such as a wearable device present on the user.

FIG. 1 illustrates a preferred embodiment of the invention, in particular a model used to assess and/or optimize the engagement of a user in an activity. A system 10 used to implement the novel method comprises both hardware and software elements. A device of the system 10 used to implement the novel method may be a mobile phone, a computer, a tablet or similar device that may be part of another device, e.g., a manufacturing machine or that may be separate. The device preferably comprises a number of hardware sensors, for example, a camera, a microphone, a speaker, a screen, a keyboard, a touchscreen or a mouse. The sensors providing data to the device may be part of a different device, such as a smart watch. The device may be configured to additionally access external resources such as remote servers.

The novel method is implemented using a model that indicates for the user which manner of delivering an instruction (e.g., an instructions supported by a motivating video) leads to optimal user engagement. The system 10 uses a speech-to-text conversion unit and a text-to-speech conversion unit and a unit configured for object recognition in images. A control module of a device used to implement the method may be configured to perform the functions of these units. The device is also configured to access a database that stores the materials belonging to the various activities, motivators and instructions of the model, such as videos, texts, images, etc.

By using a speech-to-text conversion unit and/or a text-to-speech conversion unit, the device can interact with a user who is, e.g., visually impaired or not capable of reading and/or writing. The software elements of the system 10 comprise multiple units at different levels which can also be regarded as blocks or modules of a model. A block hierarchy is preferably introduced to advantageously cluster the elements.

The embodiment of the model shown in FIG. 1 comprises multiple engagement validation blocks (VB and VC), which describe the aspects that are to be monitored in order to evaluate the engagement related to each block. The term engagement validation preferably applies to both the engagement in an activity (VB blocks) and the engagement in the motivation for an activity (VC blocks).

Another element of the model is the validation number, wherein the term number preferably may involve a complex data structure like a vector of numbers with timestamps, part of the engagement validation blocks, which is evaluated based on the criteria defined by the validation blocks. The validation number may be a score indicating the engagement of the user in an activity and/or with a motivation means or motivator.

The model also includes global blocks, which are used to monitor and/or control general aspects such as the aesthetics of the graphical user interface presented to the user (e.g., modifying the size of the buttons), the way of writing (e.g., communication style from colloquial to very formal communication) or the usage patterns (e.g., usual time of the day for interacting with the device, frequency of the interactions, etc.).

Besides the model, system 10 further comprises a control unit that analyses the model and/or receives input therefrom, in particular the engagement values captured by the model, and changes the mode of delivery of an instruction accordingly. For example, the output provided by the system 10 to the user may be changed from video-1 to video-2 to motivate the user to engage in activity B-1 or B-M, as shown in FIG. 1. The control unit may also connect to external resources, such as databases, to exchange information. The control unit initializes the model after the model is generated.

The model summaries the user's needs in terms of instruction delivery and is regularly updated. The goal of the system 10 of FIG. 1 is to induce the user to systematically try to complete an instructed activity (also called an action or task); to achieve this various motivators are used. When the user is systematically trying to complete an instructed activity, the engagement is high regardless of whether the user successfully completes the activity.

As shown in FIG. 1, the software model is organized in blocks, which are grouped in different levels. A first level splits the blocks A-1 to A-N according to the overall task, topic or field of application, such as manufacturing or maintenance. Each user may be involved in more than one topic at a time; typically up to three topics simultaneously.

Each element A has, as sub-elements, all possible activities or actions that the user may be instructed to perform and that relate to the specific field corresponding to each block A. In FIG. 1, these sub-elements are labeled as blocks B ranging from B-1 to B-M. Examples of elements B are reading a text for 2 minutes and walking for 3 minutes. Each of the activates of the elements B involves the interacting with the device directly or indirectly. An example of indirect interaction is passive sensing or monitoring of parameters while the user performs activities.

In FIG. 1, some of these elements B are marked by an asterisk “*” such as the activity B-M. Elements B marked in this way correspond to the activities the user should perform. The other activities are simply not applicable for this user at this time.

Each of the elements B has an associated validation block labeled as elements VB in FIG. 1, ranging from VB-1 to VB-M. The validation blocks describe the parameters that have to be monitored in order to determine user engagement with the associated activity. An example of a validation block VB could be, for the case of walking 3 minutes, monitoring the GPS data and the pedometer data (derived from the accelerometer) to objectively determine whether the user has attempted to complete the task and/or with how much effort the user has attempted to complete the task.

The elements B correspond to the activities themselves. Each of these elements can be presented to the user or motivated, for example supported or accompanied by a motivator, in a different way. Each element B is associated with or includes all possible motivators or motivation means (or ways of motivating the user to engage in the associated activity). In the example shown in FIG. 1, the motivators are depicted as elements C ranging from C-1 to C-P (wherein P is any real positive number) and correspond to presenting a quiz game, audio content, video content, images, interactive inputs or texts and articles. These are only examples and the motivators or motivation means associated with each element C can be chosen at will.

Each element C has an associated validation block (labeled as elements VC in FIG. 1, ranging from VC-1 to VC-P), which in this example describes the parameters that are to be monitored to determine whether the user is engaging with the motivator.

Under an element C, all possible materials corresponding to the motivator are listed together with tags that describe their content. For example, if the element C-3 corresponds to video content, the materials associated with this element are videos, and each video includes tags such as sports, competitive, family, emotional or divulgative.

Furthermore, as shown in FIG. 1, the software model also includes global blocks (elements D ranging from D-1 to D-L and element E), which control aesthetic aspects of the graphical user interface, language aspects and usage patterns, among other things. Each of the elements D includes a description of how to measure and quantify the monitored aspect. A global block, element E, is the control block. This block is responsible for the actual personalization (e.g., deciding to switch from video-1 to video-2 to improve user engagement) and communicating to external blocks. This element E has access to all other blocks.

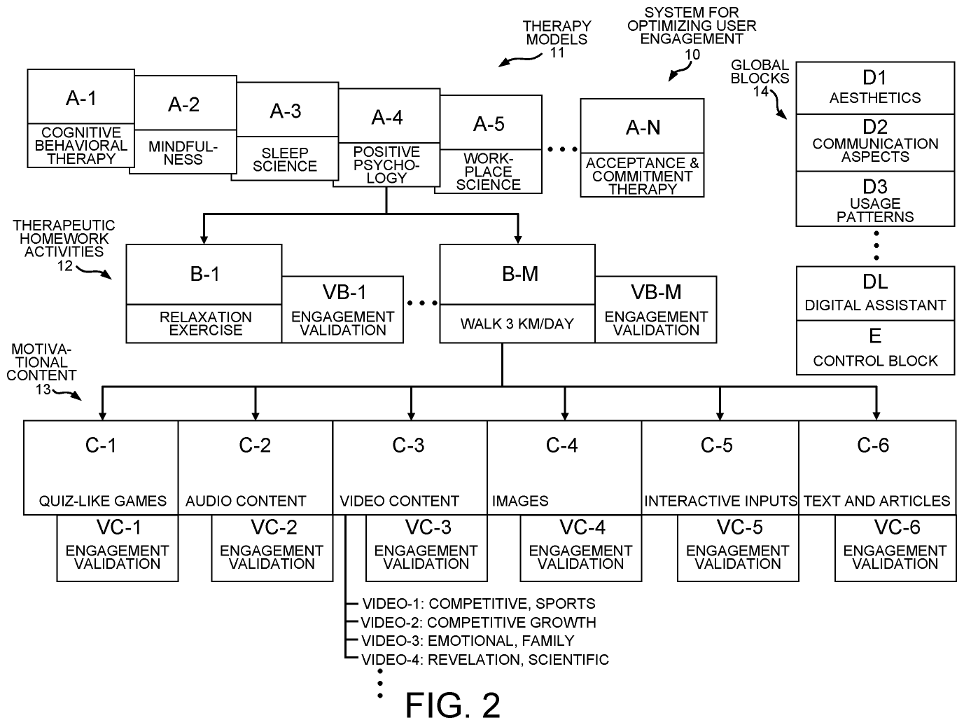

FIG. 2 shows another embodiment of system 10 and the model that uses motivators to induce the user to attempt to complete the instructed activity, which in this embodiment are associated with the field of therapies and interventions. The software model is used to administer digital therapies for example by running on the processor of a smartphone as a mobile app. The therapy models 11 of the blocks A include therapies such as mindfulness, sleep science, positive psychology, workplace science, acceptance and commitment therapies and cognitive behavioral therapy (CBT). In this embodiment, the activities of blocks B are therapeutic homework activities 12, which can include interventions. For example, activity B-1 is a relaxation exercise, and activity B-M requires the user to walk 3 km per day.

The user is motivated to engage in the activities 12 that implement the digital therapies 11 through motivational content 13, which is depicted as elements C and includes motivators such as quiz-like games, audio content, video content, images, interactive inputs and texts and articles. The embodiment of FIG. 2 also includes global blocks 14 (elements D and E) that control aesthetic aspects of the graphical user interface, language aspects and usage patterns, how to measure and quantify indicia of the user's engagement, and the personalization of the motivational content 13 to the specific user.

System 10 and the software model of FIG. 2 assess how engaged the patient is in the therapeutic homework activity 12 and determine the motivator 13 that is most effective at inducing the patient to attempt to complete the activity. The software model implements the novel method for determining and using the most effective motivator to induce the patient to engage in the therapeutic homework activity 12. First, the patient is instructed to perform the activity 12. An activity parameter is monitored to assess the engagement of the patient in performing the activity 12. The patient is exposed to a first motivator 13, which prompts the patient to perform the activity 12. A motivator parameter is monitored to assess the engagement of the patient with the first motivator 13.

The efficacy is determined of the first motivator at motivating the patient to perform the activity; the efficacy is determined by customizing the model to the patient based on the activity parameter and the motivator parameter.

The activity parameter and the motivator parameter are indicators of engagement and measure such aspects as number of attempts, attempt duration, active listening, active screen watching, perseverance in physical activities, and breathing rate and pulse rate for relaxation exercises.

The number of attempts can be used to monitor most activities, such as a physical activity, reading a document, watching a video, etc. This parameter is a simple count of the number of attempts at completing the homework.

The attempt duration can also be used to monitor most activities, such as a physical activity or reading a document. The amount of time that the patient spends trying to complete the task is measured. Where the activity is reading a document that is displayed by a smartphone or any device with an internal clock, the amount of time that the document is displayed is monitored. The presentation time is similarly monitored for other materials such as audio content, video content, images, games and interactive inputs. When a physical activity is involved such as walking, monitor the amount of time walking (determined using the accelerometer of the smartphone or wearable device) is monitored.

Active listening is monitored by verifying that the patient is attentive to the audio content, which can be difficult. For example, a patient might play audio content and then fall asleep, start talking to another person, or start watching television instead of carefully listening to the audio content. The system 10 determines that the patient is not actively listening if the amount of background noise is excessive, for example when the audio content is being played by the speakers, and the noise captured by the microphone exceeds a certain threshold. This threshold is adapted depending on the play volume and depending on whether headphones are used.

Active screen watching is monitored when the activity 12 involves watching content displayed on the smartphone screen, such as images, texts, or videos. The camera of the smartphone is used to verify that the patient is looking at the screen while the content is being displayed. The amount of active time watching can be defined as the ratio of the amount of time that the user watches the screen compared to the total time during which the content is displayed. When the patient is watching the screen, the front camera captures a close-up of the patient's face. Active watching is occurring when the patient's face is stationary and perpendicular to the camera (frontal view) and the patient's pupils are moving.

Perseverance in physical activities is monitored using sensors on the smartphone, such as GPS data and accelerometer data. These can be used to quantify steps taken and distance traveled. Heart rate data is also available from smartwatches and wearable devices to confirm that the physical activity is associated with increased effort.

Some therapeutic homework activities involve relaxation and meditation, which can also be monitored using heart rate and breathing rate of the patient. The camera and accelerometer on the smartphone can be used to measure breathing rate and heart (pulse) rate in real-time. The system monitors whether the patient's breathing rate and heart rate are decreasing while the patient is engaging in the homework activity.

The next step of the method is to compare the activity parameter to an activity engagement threshold. In addition, the motivator parameter is compared to a motivator engagement threshold. If either or both the activity parameter is below the activity engagement threshold or the motivator parameter is below the motivator engagement threshold, then the model is used to select a second motivator that is expected to increase the patient's engagement. The patient is then exposed to the second motivator.

The novel method also identifies a physician or health professional who is most likely to induce the patient to engage in the therapeutic homework activity. An attribute of the patient is identified that indicates a preference of the patient for the first motivator using the model. Based on the attribute, a support source is selected that the model predicts will likely support the patient in performing the activity. For example, support source can be a physician, a health professional, a chatbot, or an avatar.

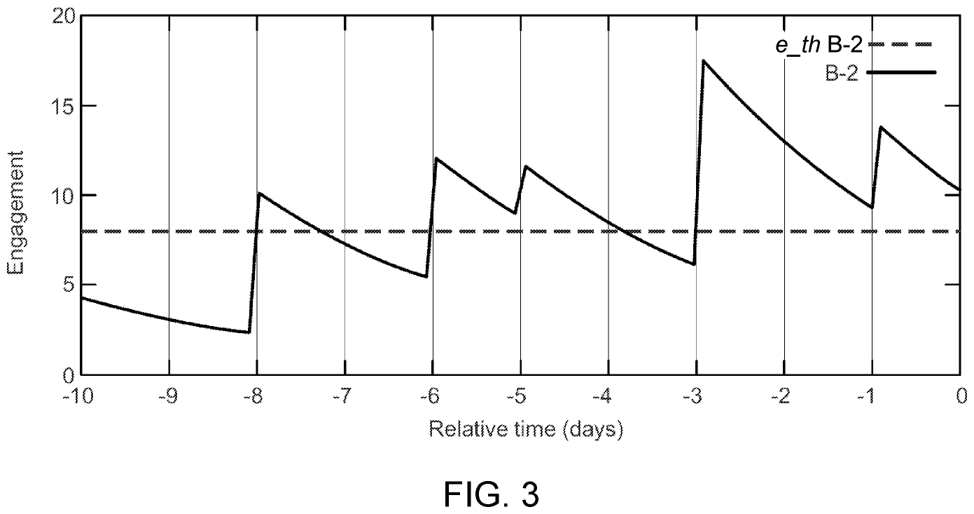

FIG. 3 illustrates an exemplary manner of measuring user engagement in an activity. In one embodiment, the model is configured to assess the engagement of the user in an activity or motivator by performing the calculations described below. The term user engagement in this example concerns only the level of involvement of the user and the predisposition of the user towards carrying out the activity that has been recommended. User engagement is not based on the completion of the activity or the outcome for the user as a result of engaging in the activity. In other words, a user is highly engaged with an activity at the moment that the user systematically performs steps in an attempt to complete the activity (regardless of whether the activity is every completed).

For example, if a user is instructed to perform a specific activity for 20 minutes every day, a high engagement with this activity is achieved when the user performs the activity every day, at least once a day and spends a minimum of 10 minutes engaging in the activity every day. Systematically attempting but failing to achieve the goal may indicate that the level of the activity is not appropriate for this user, for example the user may not yet be competent to perform the given task.

The way of measuring user engagement depends on the nature of the activity to be carried out. The manner of measuring the user engagement is automatic, without having explicitly to ask the user to provide an input or without the user even noticing. For most activities, monitoring the number of attempts and the time length of the attempts (or amount of completion, depending on the activity) already provides valid insights about user engagement.

How insights about user engagement are gathered is further described below. Assuming that the engagement in each individual attempt can be measured and quantified, the activity engagement e_{B}(t) (wherein the activity corresponds to an element B in the models of FIGS. 1-2) can be modeled as shown in FIG. 3. The activity engagement is modeled as a variable, whose minimum value is 0 (no engagement). The higher the value, the higher the engagement. An activity engagement threshold value e_th is defined as well; this value must be larger than zero.

Each attempt at completing the activity is preferably incorporated into the activity engagement as the function f(t). This function f(t) should be zero before the first attempt. The function then steeply increases up to a local maximum value at the moment of an attempt and then gradually decreases over time (towards 0). The term t may be regarded as referring to time, with t=0 referring to the present time. Negative values of t indicate past events and positive values of t indicate future events.

This function f(t) can be expressed as:

$$f ( t ) = Au ( t + {t}_{0} ) {e}^{- \frac{t + {t}_{0}}{τ}}$$

where t₀ denotes the time elapsed since the attempt took place until now, τ is a time constant, A is the intensity of the attempt (this is further elaborated below) and u(t) is the heavyside function defined as u(t)=0 if t<0, otherwise 1.

To combine multiple events, the total engagement e_{B}(t) is evaluated as the addition of all individual activity contributions fᵢ(t):

$${e}_{B} ( t ) = {\sum }_{i} {f}_{i} ( t )$$

This function can be used to plot summaries, for reporting purposes. To evaluate the current engagement, it is necessary to evaluate only the current value (this is, t=0).

$${e}_{B} ( t ) = {\sum }_{i} {f}_{i} ( 0 )$$

From all past events, only those whose contribution is large enough are considered; the rest can be neglected. For instance, the contribution must be larger than 0.01e_th; formally fᵢ(0)>0.01e_th.

The value of the different parameters must be set accordingly with the goals or instructed activities. One way to determine τ is to assume high engagement for a long period of time; then τ must be set so that the engagement is higher than a certain value, e.g., 2e_th.

This can be illustrated with an example: the instructed activity is performing a maintenance work routine three to four times a week that includes walking 10 minutes to fetch a component.

Every time the user starts walking, the contribution is set to A=1.5; this value increases the longer the user walks or the more steps the user takes. If the user walks around for 10 minutes, the contribution is increased to A=2.5. The threshold is also e_th=2.5 (exactly the same as the contribution of a single day because it is pursued that the user engages regularly).

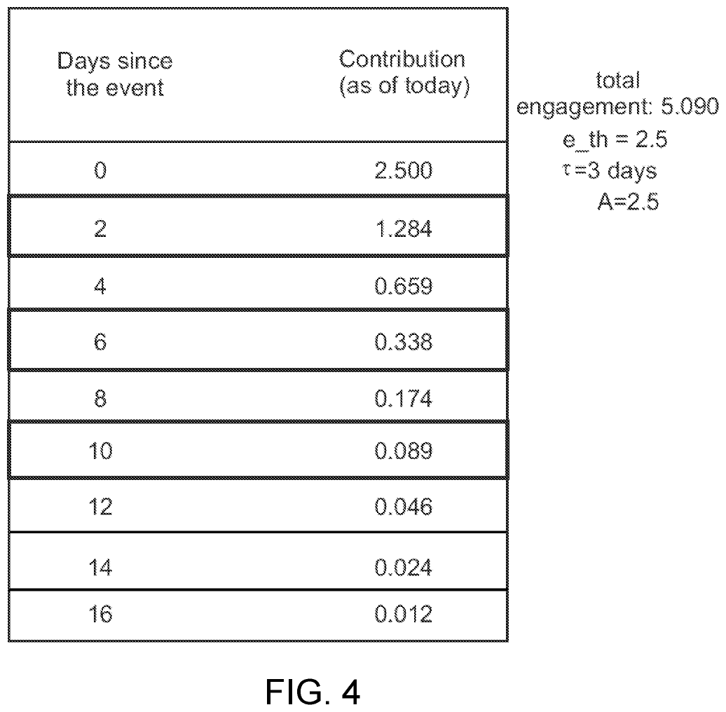

In such a case, because the engagement has been high for a long time, there will be a very large number of past events. The number of past events is so large that the contribution of the oldest will already be below the threshold. Considering all this, defining a τ of three days considers the events of the (approximately) last two weeks and, if the user regularly walks the required distance, the aggregated contribution is about 2e_th, as shown in the table of FIG. 4.

The exponential function described above is just an example. Different calculations for the function f(t) may be used. A simpler example is f(t)=1 when t ϵ[−τ0, −τ0+τ], and otherwise 0.

In the following an example is given of quantifying the intensity of an attempt of the user to perform an instructed task. In the previous example, the amplitude A was evaluated based on the attempt itself and the time spent walking or the number of steps taken (validation for this activity).

These two validation elements, the attempt itself and the amount of completion or the time spent, are accurate criteria for most activities, but generally these are not representative enough.

For instance, in the case wherein a video is presented to the user via a smartphone, the user may leave the phone unattended and do something different. Without any other input, this situation would be considered as high engagement, when it should not be.

Next, examples of objective criteria to measure engagement are listed. These criteria are accurate for both activities (elements B) and motivators (elements C), as shown in FIGS. 1-2. For instance, reading could be an instructed activity (corresponding to an element B such as reading a chapter of a book), but it can also be a motivator (corresponding to an element C such as a short text explaining why is it important to perform the instructed activity). The following exemplary criteria may be analyzed by the model to quantify the intensity of an attempt of the user to perform an instructed task.

The number of attempts. This applies to most activities: physical activity, such as walking or fetching, operating a lever, reading a document, watching a video, etc. This metric corresponds to simple counting of the number of attempts the user makes at completing the instructed activity.

Attempt duration. This applies to most activities: physical activity, reading a document, etc. The amount of time that the user spends trying to complete the activity is measured. An attempt at reading a document (displayed for example by a smartphone or any device with an internal clock) would be quantified by monitoring the amount of time that the document is displayed; similarly for any other material such as audio, video, images, games and interactive inputs.

When a physical activity is instructed (e.g., walking), the amount of time the user spends walking (corresponding to an elapsed time between start of the walk and end of the walk) is monitored. The measured time, e.g., a number in seconds, preferably is translated into a different value, for example into a score in a range from 0 to 10. This translation can be performed in different ways, for instance, by normalizing the measured time by a fixed value that expresses the amount of time that is expected to be used by a user to complete the task. And this value can saturate at a maximum value. Formally, a1=Δt/2·T0 if Δt<2·T0, otherwise 1, where Δt expresses the measured time and T₀ the expected time required to complete the task.

Active screen watching. When the activity involves attentively watching something displayed on a screen (images, texts, videos, etc.), it can be verified that the user is looking at the screen on which the content is output. This requires a camera. The amount of active time watching can be defined as the ratio of the amount of time that the user watches the screen Δt compared to the total time during which the content is reproduced T₀; this is especially suited for videos.

$${a}_{2} = \frac{Δ t}{{T}_{0}}$$

For images and texts, it may be relevant to compute the time during which the user actively watches the screen for each image/text fragment Δtᵢ, and normalize this measured value with an expected value for that image/text fragment T₀ᵢ; and then average over the number of images or fragments N. Further criteria may involve a minimum time per image/fragment, a saturation as in the attempt duration, etc.

$${a}_{2} = \frac{1}{N} {\sum }_{i = 1}^{N} \frac{Δ {t}_{i}}{{T}_{0 i}}$$

Active screen watching can be evaluated with a face tracker and a camera. The interaction of a user with a device often involves interaction of the user with a smartphone or tablet, which regularly include a front camera. When the user watches the screen, the front camera captures a close-up of the face of the user; the face is then perpendicular to the camera (frontal view). In a first approximation, active screen watching (At in the examples above) is detected to occur when the camera captures a front view of the user's face, or in another embodiment, the user's pupil may also be tracked. The face is identified, and this can be done with a face tracker. Face trackers do not identify only the face contour but also the landmarks and inner contours on the face, such as the eyes, upper and lower eyebrow bounds, nose bridge, nose bottom or the upper and lower bounds for both the upper and lower lip. With all this information, the orientation of the face can be evaluated. When the detected orientation is frontal, then the user is actively watching. Otherwise (different orientation, not enough landmarks detected or no face detected at all), the user is not watching the screen.

Active listening. Verifying that a user is listening to audio content is more challenging than verifying active screen watching. A user may play the audio and fall asleep, start talking with another person or watching TV as opposed to carefully listening to the audio. A necessary (yet not sufficient) condition for active listening is a low amount of background noise. Thus, quantifying background noise may be used to quantify attentive listening. When the audio is played by the speakers, the noise captured by the microphone must not exceed a certain threshold. This threshold may be adapted depending on the play volume and depending on whether headphones are used.

Physical activities can be monitored with the use of sensors, for example sensors present on wearable devices worn by the user. In particular, GPS and pedometer (accelerometer) data can be used to objectively quantify walking activity and distance travelled. Additionally, the heart rate of the user may also be monitored, which can be used to confirm that walking is associated with an increased effort.

Generally, physiological parameters of the user may be monitored, for example the breathing rate and the pulse rate. These parameters may be measured directly or for example extracted from a captured video of the user (e.g., chest movements relating to breathing rate). These physiological parameters may be used by the model to select a suitable motivator. For example, a user who has a high pulse rate and breathing rate may respond better to calming motivators, and a user that has a low pulse rate and breathing rate may be stirred up by a more stirring motivator. The model and novel method may use measurements data stemming from sensors present on external devices, for example wearable devices worn by the user or other devices, such as a tablet, a smartphone or a laptop, etc. Thus, sensors for monitoring physiological parameters of the user may be present in or at the device itself or may be present on other, external, devices.

When performing activities, the position of the user may change, and the user may even lay down. In such a case, the device, for example a smartphone, can be placed on the user's chest, and both the pulse rate and the respiration rate can be measured from the accelerometer sensor. If the user sits, the heart rate and the respiration rate can be remotely monitored with a camera, e.g., by placing the phone in front of the user pointing towards him or her.

Regardless of the sensor used, a waveform tracking the user breathing is available. If the waveform relates to the chest volume and not to the change of the chest volume, the breathing stages can be identified by computing the derivative of this raw breathing waveform. Inhalations are captured as positive derivative values, exhalations are captured as negative derivative values, and apneas are captured as very small amplitude derivative values (close to zero). Because breathing can be consciously controlled, an instantaneous breathing waveform is required; a value expressing the average breathing rate may not be enough.

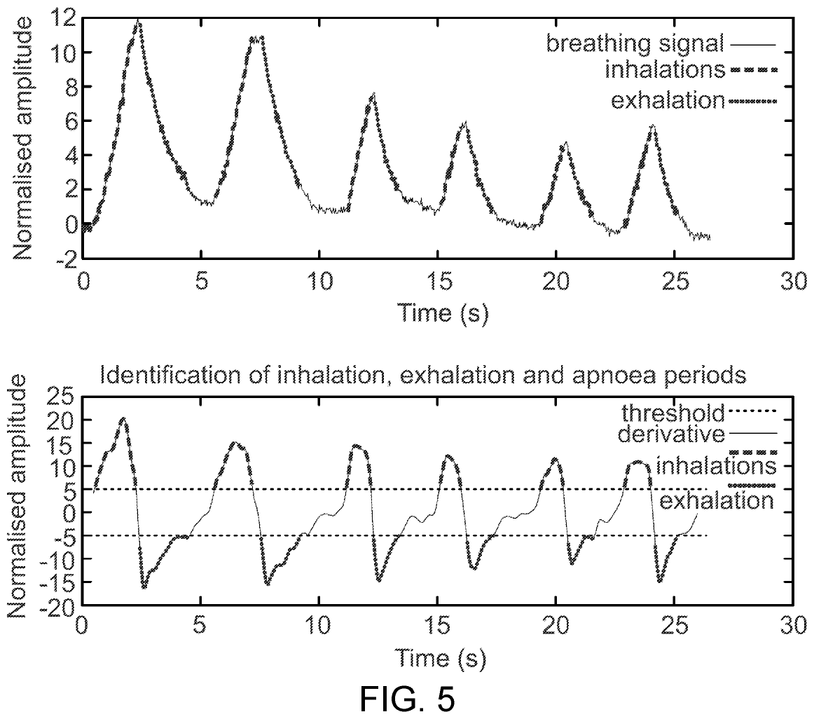

FIG. 5 illustrates instantaneous breathing waveforms this with an example. In this case, the breathing waveform is obtained with a camera. The waveform relates to the chest volume (upper plot). The derivative is evaluated with the kernel [−4, −3, −2, −1, 0, 1, 2, 3, 4]; the derivative signal (very noisy) is smoothed with a median filter and a moving-average filter, both of length 9. The filtered signal (the one displayed in the lower plot of FIG. 5) is then compared to a threshold to identify the increasing (inhalations) and decreasing (exhalations) intervals. The segments of the signal belonging to none of these are then apneas. The intrinsic filter delays have been compensated before plotting for the sake of clarity.

The movement of the heart is not directly controllable by the user. However, if properly engaging in some activities, such as the user standing still to read a text, it is expected that the average heart rate of the user decreases or remains within a certain range of values. Furthermore, certain patterns in the heart rate or the measured waveform may be indicative of a relaxed state, such as Respiratory Sinus Arrhythmia (synchronous variation of the instantaneous heart rate and respiration). All of these features can be used as an additional criteria to verify user engagement in an instructed activity.

In the following, an example is given of distinguishing between user preference, e.g., the user subjectively prefers texts, and user needs, e.g., the user objectively needs to be exposed to videos to be motivated to perform an instructed activity. Distinguishing between user preferences (that which is preferred or consciously chosen by a user) and user need (that which is effective, receives the most attention or is mostly used by the user) is an important aspect of the novel method. The user preference is a subjective choice and therefore prone to biases (voluntarily or involuntarily). For instance, a user may claim to enjoy reading (user preference), but when prompted with a text, the user may quickly skip it and stop at the drawings. Therefore, it may be easier reaching to that user by presenting images and videos to the user (user need) instead of texts (user preference). Properly distinguishing between user preferences and user needs requires continuous user monitoring while the user is interacting with the device and attempting to complete the instructed activity.

User preferences are identified by directly asking the user for his or her opinion or preference. User needs are identified by observing the behavior of the user when prompted with different kinds of inputs, for example, using a monitoring unit with sensors.

The user preference can be used to initialize the model, as a starting point. For example, based on the user stating that he or she prefers texts, the model initially will select motivators in the form of texts. Relying on the measurements described above, preferably the model is subsequently customized to objectively maximize the engagement of the user in the different activities that the user is instructed to perform. Thus, based on the acquired data capturing the behavior of the user, the model may over time change the type of motivator selected, e.g., images instead of texts. In principle, the model could be initialized to any state and, after some interactions with the user, the same end state would be reached.

Nevertheless, in practice, it is advisable to start from a known state and, if possible, already close to the final desired state.

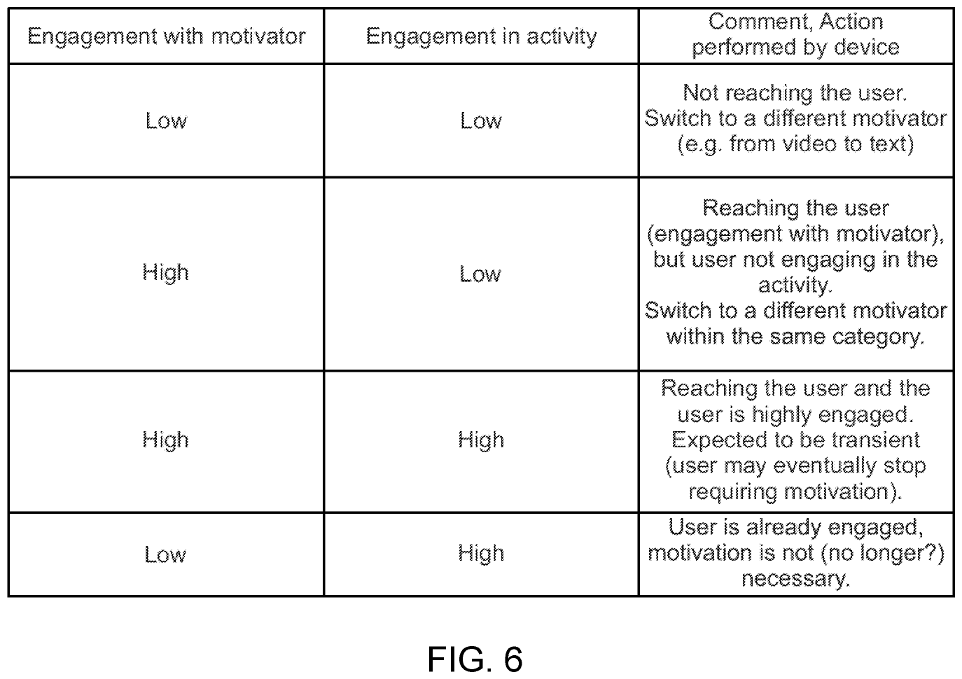

As discussed above, two different sorts of engagements are to be distinguished: engaging in the activities themselves (instructed activities of elements B, e.g., walking) and engaging with the preparation or the motivation (the elements C, the means of reaching B, e.g., the text why it is required to go and fetch a component). The ultimate goal is to induce the user to engage with the element B. To reach this goal, the elements C are optimized. In this context, engaging with element B is understood as systematically attempting to complete the activity corresponding to element B, but not necessarily actually completing it. Depending on how the user is engaging in these two, different actions may be recommended by the model. The table of FIG. 6 shows some examples.

FIG. 6 shows several parameters used by the model to determine the intensity of several attempts at completing an instructed activity. The system and model are configured to generate an attempt intensity score for each attempt that a user makes at completing an instructed activity. This allows the model to quantify how hard the user is trying to complete the instructed activity.

In this example, a user is instructed to do a 30-minute work routine every day (corresponding to an element B). The user claims to feel comfortable with explanatory texts, so initially the user is prompted to perform the routine with texts that describe why the work routine is important (corresponding to an element C). When prompted with the text, the user reads it (active reading) in half the expected time (3 minutes), as indicated in the sample calculation below.

A1_C1=1.3 min/3 min=0.43.

The intensity of this attempt is low, and therefore the engagement in this element C is low. In this example, the engagement in the element C is computed as the average of the last three events. Furthermore, the user does not attempt to complete the work routine, but instead stops after reading the text.

On a second day, the user is prompted with a shorter text (2 minutes) which also has an image, but a similar outcome results.

A2_C1=1.1 min/2 min=0.55.

However, the user stops at the image and actively watches it for 10 seconds, which is the expected engagement time for this image. Therefore,

A2′_C1=10s/10s=1.00.

On average, the user is exhibiting an average engagement with the element C of 0.49 (low, target is 0.85) and an engagement of 0 with the element B (no attempts). Because both engagements are low, the user or patient has not been reached. A different element C should be explored in order to enhance the overall user engagement. The next element C could be chosen at random or, because there is already the insight of the image, the model switches to element C2, a video based on scientific analysis.

When prompted with the video content of element C2, the user actively watches the first half, but then fast forwards through the second half.

A3_C2=1.6 min/2 min=0.80.

This engagement value is higher than any of the previous C elements, yet not high enough. This suggests that a video is a better way of reaching this user, but the content is not interesting enough to retain the user's engagement throughout the entire video. And the user also does not perform the instructed work routine either.

During the next engagement attempt, a different video is shown to the user. Instead of showing a science-based video, the new video highlights the benefits of performing the work routine, focusing on improvements in the operation of the machine and praise from colleagues. This time, the user actively watches the whole video and attempts to perform the work routine.

A4_C3=2 min/2 min=1.00.

The average motivation engagement is still low (0.78), yet it is increasing. And so is the activity engagement. Following this approach, the contents that reach the user and result in the user engaging in the activity B can be identified. Note that the most effective motivators do not necessarily coincide with the conscious user choice (user preference).

Besides the instantaneous engagement, it is also relevant to look at the change in engagement. When, after a period of time of engagement improvement in a particular activity B, a saturation period can be reached. In that event, a higher module of the software model is notified that it may be advisable to change the instructed activity.

Thus, in one embodiment, the system 10 is configured to detect changes in the attempt intensity score over a time interval. The time interval can for example span two attempts so that the change in the attempt intensity score may indicate that the attempts of the user to complete an activity are getting more or less intense.

Besides optimizing the motivators 13 (elements C) used to induce the user to engage in the activities 12 (elements B), there are other engagement-related aspects that can be optimized. These refer to the way of interacting with the smartphone, device or machine or the application running thereon. Multiple aspects can be optimized, some of which are described below.

The manner in which the user interacts with the application can be monitored as well and used as input for further customization. In one embodiment, the device is configured to analyze the interaction of the user with the device, for example, typing patterns of the user on a screen of the device, and to infer characteristics of the user therefrom. The characteristics of the user determined in this are entered into the model. For example, the device may switch from interacting with the user in writing to voice-based communication if the analysis determines that the user cannot easily read and/or write text. In this case, the device may use a speech-to-text conversion unit and/or a text-to-speech conversion unit to interact with the user.

One aspect that can be optimized is the font size and thus accessibility of information presented to the user. For example, displayed elements may be too small for a user to see. Users may have different visual acuity but also use devices with varying screen sizes. Especially when smartphones are used, which have relatively small displays, it is common for users to zoom in to better see details. The detection of frequent zooming in or multiple missed taps around a target may suggest that the current font size is too small for the user. Thus, if such a detection is made, the control unit may control the device to increase font and button sizes and/or use a high-contrast theme for the graphical user interface presented to the user.

Furthermore, it is possible to determine how much the user relies on examples and additional information, for example, by tracking how much the “learn more” (or similar) button is clicked by the user. This provides insights about how much information should be presented to the user at one time. Some users may prefer first to read a comprehensive description before performing an activity, while other users may prefer to start with almost no background information and progressively learn by interacting with built-in examples. These insights can be used to adjust the amount of information presented for new activities in order to make it easier for the user to engage with the device to complete these activities.

Typing patterns of the user may also be monitored. Slow typing, possibly combined with frequently making mistakes when typing (frequent use of backspace delete), may be an indication of difficulties in typing. If this is detected, the device may offer enabling speech-to-text support to allow the user to dictate instead of typing. When audio-based interactions are offered, a digital assistant may be offered for voice-based interactions between the user and the device.

As part of the global tuning, notifications may also be customized. In particular, reminders may be sent to the user if there were no interactions with the device or application for a certain time period. These notifications may be sent during the time intervals in which the user normally mostly interacts with the application, for example, between 8 AM and 10 AM of every day. The model can also monitor whether the user reacts to notifications or chooses to ignore them.

When the user inputs textual information, besides analyzing the typing patterns, the text itself can be analyzed. Machine learning-based algorithms can be used to infer certain characteristics (e.g., age estimation, education level, etc.), even though simpler rule-based algorithms can be used as well. For instance, aspects that can be identified are the time of communication (part of the day, day of the week, etc.), the communication length (short or long messages), the frequency of the communication (e.g., number of times per week) and the vocabulary (technical or general, using abbreviations, slang words, spelling mistakes, etc.). These, however, are only examples.

The system 10 and software model shown in FIGS. 1-2 is optimized towards finding the most effective way to reach the user, in other words, how to approach the user to maximize his or her engagement in different activities. By combining all engagement information from all blocks, it is possible to infer a general user profile (profile summary) that can subsequently be used to customize a chat bot (a virtual support system).

In other words, the device or machine that implements the novel method can be configured to generate a user profile based on acquired data and/or parameters relating to the user using the software model. The user profile can be a list of words that characterize the user, or a list of words plus a score indicating the significance of that word to the user. It can be created from the engagement values from the engagement validation blocks VB and VC. For instance, by simply creating a number of lists with all tags (from all blocks) and adding the engagement value of each element to the corresponding tags, the predominant tags for a user can be identified. These tags constitute (a first version of) the profile summary. Plausibility checks can be added to ensure that the model generates accurate profiles. For example, before the first profile summary is output, a minimum number of model updates are calculated, or a minimum distance between the dominant tags and the other tags is determined, or the values for a defined scale are normalized. The user profile can be augmented with insights gathered from global tuning. For example, if a user's interest in additional information and “learn more” is identified, an associated tag could be “curious”.

A virtual support system can be tuned using the list of words that describe the user. The list has been determined from those aspects, such as motivators, that are most effective for the particular user and not from what the user claims. Not only the aspect (e.g., the appearance of the avatar, background of the avatar, etc.) but also the communication style (e.g., formal versus colloquial communication) can be tailored to the needs of the particular user. For instance, from the detected language patterns used by the user, the chat bot can be adapted to write in the same way, e.g., using the same kind of abbreviations that the user employs.

Initially the software model of system 20 is generic and must be initialized. The guidelines for initialization can be provided by the user directly (e.g., the user entering user preference), by another person, fetched from an external resource (electronic records), or initialized by default to a value or inferred from an onboarding test period.

Regardless of the initial state of the software model, frequent interactions of the user with the machine, device or application that result in frequent model updates will ultimately lead to the same result, as the model will over time learn to identify the characteristics of the user and be increasingly capable of distinguishing subjective user preferences from objective user needs. However, it is advisable to initialize the model into a known state or into a state in which the user would already feel comfortable, e.g., a state in which the settings reflect the user preference.

Although the present invention has been described in connection with certain specific embodiments for instructional purposes, the present invention is not limited thereto. If certain elements or features of the invention are disclosed herein in a particular combination or in the context of a particular embodiment, these elements or features may also exist in isolation or in different combinations or in the context of a different embodiment. Accordingly, various modifications, adaptations, and combinations of various features of the described embodiments can be practiced without departing from the scope of the invention as set forth in the claims.

1-16. (canceled)

17. A method for assessing how engaged a user is in an activity, comprising:
instructing the user to perform the activity;
monitoring an activity parameter to assess an engagement of the user in performing the activity;
exposing the user to a first motivator, wherein the first motivator prompts the user to perform the activity;
monitoring a motivator parameter to assess the engagement of the user with the first motivator;
determining an efficacy of the first motivator at motivating the user to perform the activity using a model customized to the user based on the activity parameter and the motivator parameter;
comparing the activity parameter to an activity engagement threshold;
comparing the motivator parameter to a motivator engagement threshold;
if either or both the activity parameter is below the activity engagement threshold or the motivator parameter is below the motivator engagement threshold, using the model to select a second motivator; and
exposing the user to the second motivator.

18. The method of claim 17, further comprising:
updating the model with a subsequently monitored activity parameter.

19. The method of claim 17, further comprising:
determining a change in the activity parameter monitored at a current time compared to the activity parameter monitored at a past time.

20. The method of claim 17, further comprising:
identifying an attribute of the user that indicates a preference of the user for the first motivator using the model.

21. The method of claim 20, further comprising:
selecting based on the attribute a support source that the model predicts is likely to support the user in performing the activity, wherein the support source is selected from the group consisting of: a physician, a chatbot, and an avatar.

22. The method of claim 20, further comprising:
generating a virtual support system that the model predicts is likely to support the user in performing the activity.

23. A system for assessing how engaged a user is in an activity, comprising:
an output unit that instructs the user to perform the activity;
a monitoring unit that assesses an engagement of the user in performing the activity by monitoring an activity parameter, wherein the output unit exposes the user to a first motivator that prompts the user to perform the activity, and wherein the monitoring unit assesses the engagement of the user with the first motivator by monitoring a motivator parameter;
an evaluation unit that determines an efficacy of the first motivator at motivating the user to perform the activity by using a model customized to the user based on the activity parameter and the motivator parameter; and
a comparison unit that compares the activity parameter to an activity engagement threshold and that compares the motivator parameter to a motivator engagement threshold, wherein the evaluation unit uses the model to select a second motivator if either or both the activity parameter is below the activity engagement threshold or the motivator parameter is below the motivator engagement threshold, and wherein the output unit exposes the user to the second motivator.

24. The system of claim 23, wherein the evaluation unit updates the model with a subsequently monitored activity parameter.

25. The system of claim 23, wherein the evaluation unit determines a change in the activity parameter monitored at a current time compared to the activity parameter monitored at a past time.

26. The system of claim 23, wherein the evaluation unit uses the model to identify an attribute of the user that indicates a preference of the user for the first motivator.

27. The system of claim 26, wherein the evaluation unit selects based on the attribute a support source that the model predicts is likely to support the user in performing the activity, and wherein the support source is selected from the group consisting of: a physician, a chatbot, and an avatar.

28. The system of claim 26, wherein the system generates a virtual support system that the model predicts is likely to support the user in performing the activity.

29. A method for assessing how engaged a user is in a digital mental health intervention, comprising:
exposing the user to a first motivator, wherein the first motivator prompts the user to perform the intervention;
monitoring an intervention parameter to assess an engagement of the user in performing the intervention;
monitoring a motivator parameter to assess the engagement of the user with the first motivator;
personalizing an intervention delivery model to the user based on the intervention parameter and the motivator parameter;
determining an efficacy of the first motivator at motivating the user to perform the intervention using the intervention delivery model;
comparing the intervention parameter to an intervention engagement threshold;
comparing the motivator parameter to a motivator engagement threshold;
if either or both the intervention parameter is below the intervention engagement threshold or the motivator parameter is below the motivator engagement threshold, using the intervention delivery model to select a second motivator; and
exposing the user to the second motivator.

30. The method of claim 29, further comprising:
instructing the user to perform the intervention, wherein the user is instructed to perform the intervention before the first motivator prompts the user to perform the intervention.

31. The method of claim 29, wherein the first motivator is selected from the group consisting of: watching a motivational video, listening to a motivational audio tape, engaging in a quiz-like game, and reading an explanation of how the intervention will benefit the user.

32. The method of claim 29, wherein the first motivator is a video shown to the user that explains how the user will benefit from performing the intervention.

33. The method of claim 29, further comprising:
updating the intervention delivery model with a subsequently monitored intervention parameter.

34. The method of claim 29, further comprising:
determining a change in the intervention parameter monitored at a current time compared to the intervention parameter monitored at a past time.

35. The method of claim 29, further comprising:
identifying an attribute of the user that indicates a preference of the user for the first motivator using the intervention delivery model.

36. The method of claim 35, further comprising:
selecting based on the attribute a support source that the intervention delivery model predicts is likely to support the user in performing the intervention, wherein the support source is selected from the group consisting of: a health professional, a chatbot, and an avatar.

37. The method of claim 29, further comprising:
generating a virtual support system that the intervention delivery model predicts is likely to support the user in performing the intervention.

38. The method of claim 29, further comprising:
selecting a particular health professional who the intervention delivery model predicts is best suited to supporting the user in performing the intervention.
